(19) United States
(12) Patent Application Publication
(10) Pub. No.: US 2010/0145373 A1
(43) Pub. Date: Jun. 10, 2010

(54) NAIL DRILL

(76) Inventor: Ruth Alon, Michmoret (IL)

Correspondence Address: RATNERPRESTIA, P.O. BOX 980, VALLEY FORGE, PA, 19482, US

(21) Appl. No.: 12/632,035

(22) Filed: Dec. 7, 2009

Related U.S. Application Data
(60) Provisional application No. 61/201,044, filed on Dec. 5, 2008.

(51) Int. Cl. A61B 17/32; A61N 1/30

(52) U.S. Cl. 606/169; 604/20

(57) ABSTRACT

Apparatus is provided for drilling a hole in a nail of a subject. A drill bit has a nail-contacting surface for contacting the subject's nail. A drill control unit is configured to drill a hole in the subject's nail by moving the nail-contacting surface in a back-and-forth motion, while the nail-contacting surface is in contact with the subject's nail. Other applications are also described.

### CROSS REFERENCES TO RELATED APPLICATIONS

The present application claims priority from U.S. Provisional Patent Application 61/201,044, entitled “Nail drill,” to Alon, filed Dec. 5, 2008, which is incorporated herein by reference.

### FIELD OF EMBODIMENTS OF THE INVENTION

Some applications of the present invention generally relate to external medical apparatus. Specifically, some applications of the present invention relate to drilling a subject's nail.

### BACKGROUND

Onychomycosis is a fungal infection that causes the toenails and/or fingernails to thicken, discolor, and split. The prevalence of onychomycosis in the United States population as a whole is 13%, onychomycosis being more prevalent in the elderly (60%). Onychomycosis can result in permanent nail deformity. The disease has a significant impact on the patient's quality of life (e.g., due to concern regarding the appearance of one's toenails and fingernails, and pain associated with wearing shoes, walking and sports activities).

U.S. Pat. No. 4,990,134 to Auth describes an abrasive tipped rotating cutting tool for use in removing abnormal deposits within a patient's vessels. The tip is covered with a material such as diamond grit, and is described as being rotated at high speed to pulverize any abnormal deposits contained within the vessel. In an embodiment, the tip is ellipsoidal in shape, and the coarseness of the abrasive material on the tip is varied from most coarse, adjacent the distal end of the tip, to finest, adjacent the portion of the tip which has the widest diameter. The Auth patent states that preferably, there is no abrasive on the widest part of the tip in order to prevent the tip from injuring a patient's vessel in the event that the tip is allowed to remain in one place for an extended period of time. In addition, there may be radial openings formed in the tip which permit water to be pumped therethrough in order to act as a lubricant for assisting in the cutting action of the tip. The tool is described as using either an atraumatic tip or a preformable guide wire to guide the cutting tip through a patient's vessel.

U.S. Pat. No. 4,180,058 to Brem describes a method for treating pathological conditions of the nail, particularly onychomycosis. The method comprises the breaching of the protective keratin of the nail to form an opening therein, placing a caustic-keratolytic agent in the opening to enlarge it, and treating the nail through the opening with topical therapeutic agents for the pathological condition being treated, for instance, an antifungal agent for onychomycosis. In addition, thick calluses and painful plantar warts are described as being removed with modifications of this method.

U.S. Pat. No. 6,572,580 to Feldman describes a kit including a patch laden with topical anti-fungal medication and a set depth nail notcher for cutting a notch of predetermined depth in a nail plate, comprising a handle having a smooth flat surface, a longitudinal cutting edge protruding from the smooth flat surface to a height equal to the predetermined depth of the notch. A method is described for treating nail fungus, especially toenail fungus, comprising cutting a notch to a predetermined depth in a nail of a toe or finger infected with fungus and applying a topical anti-fungal medication to the toe or finger from a patch through the notch.

PCT Publication WO 06/104600 to Jamison describes a nail drill configured to create a hole in a nail, such as a fingernail or toenail. The nail drill has a shaft extending from a handle. A tip is disposed on an end of the shaft not coupled to the handle and has one or more cutting sections configured to cut into a nail. The nail drill is described as being utilized for relieving a pressurized area underneath a nail, such as bodily fluid that may have collected due to a prior trauma or other condition. The method for relieving the pressurized area includes positioning the tip onto an outer surface of the nail. The shaft may be rotated so that the tip turns and cuts a hole in the nail. The shaft is rotated until a hole is created through the nail, enabling the bodily fluid to pass from the pressurized area through the hole, thereby relieving the painful condition.

The following patents may be of interest:

U.S. Pat. No. 7,135,194 to Birnbaum

U.S. Pat. No. 6,664,292 to Bogart

U.S. Pat. No. 6,015,418 to Wymond

The following articles may be of interest:

“Toenail Fungus: Drill to Kill,” Harvard Health Letter Mar. 1, 2007

“Progression and Recurrence of Onychomycosis,” by Scher et al., available on Medscape.

### SUMMARY OF EMBODIMENTS

In some embodiments of the present invention, the drill bit of a drill is used to drill a hole in a subject's nail. A nail-contacting surface of the drill bit is moved in a back-and-forth motion, while the nail-contacting surface is in contact with the subject's nail. Typically, the back-and-forth nature of the motion of the nail-contacting surface prevents damage to the nail-bed, which may otherwise be caused by the drilling. When the nail-contacting surface makes contact with the flexible tissue of the nail-bed, the nail-contacting surface simply moves the tissue of the nail-bed, but does not cut through the tissue. However, when the nail-contacting surface is in contact with the nail, which is generally rigid, it drills through the nail.

In some embodiments, a skin-contact sensor is used to detect contact between the nail-contacting surface and the nail-bed. The drilling is automatically stopped in response to the skin-contact sensor detecting contact between the nail-contacting surface and the nail-bed.

In some embodiments, the hole is drilled in the nail to relieve pressure associated with a blood-blister underneath the nail. Alternatively, one or more holes are drilled to facilitate treatment of onychomycosis. For example, a substance may be administered to the nail-bed via the holes for treating the onychomycosis.

There is therefore provided, in accordance with an embodiment of the present invention, apparatus for drilling a hole in a nail of a subject, including:

a drill bit having a nail-contacting surface for contacting the subject's nail; and

a drill control unit configured to drill a hole in the subject's nail by moving the nail-contacting surface in a back-and-forth motion, while the nail-contacting surface is in contact with the subject's nail.

In an embodiment, the nail-contacting surface is flat.

In an embodiment, the control unit is configured to execute 2-60 cycles of the back-and forth motion per second.

In an embodiment, the apparatus further includes a skin-contact sensor for detecting contact between the nail-contacting surface and a nail-bed of the subject.

In an embodiment, the apparatus further includes a substance for treating onychomycosis, two or more electrodes, and an iontophoresis control unit,

the drill control unit being configured to drill two or more holes in the subject's nail, and

the iontophoresis control unit being configured to iontophoretically drive the substance into a nail-bed of the subject by driving the electrodes to drive a current via the holes.

In an embodiment, the drill control unit is configured to move the nail-contacting surface in the back-and-forth motion by repeatedly (a) moving the nail-contacting surface in a first direction by an angle that is greater than 30 degrees, and (b) moving the nail-contacting surface in a second direction opposite to the first direction by the angle that is greater than 30 degrees.

In an embodiment, the drill control unit is configured to move the nail-contacting surface in the back-and-forth motion by repeatedly (a) moving the nail-contacting surface in a first direction by an angle that is less than 360 degrees, and (b) moving the nail-contacting surface in a second direction opposite to the first direction by the angle that is less than 360 degrees.

In an embodiment, the apparatus further includes anti-fungal and anti-bacterial agents for administering to a nail-bed of the subject via the hole.

In an embodiment, the apparatus further includes a finger sheath that contains a substance for treating onychomycosis, the sheath being configured to administer the substance to a nail-bed of the subject via the hole, by being placed on the subject's finger over the hole.

In an embodiment, the apparatus further includes an adhesive bandage that contains a substance for treating onychomycosis, the bandage configured to administer the substance to a nail-bed of the subject via the hole, by being adhered to the subject's finger over the hole.

In an embodiment, the apparatus is configured to drill a hole having a diameter of 0.1 mm to 2.0 mm in the subject's nail.

In an embodiment, the apparatus is configured to drill a hole having a diameter of 0.5 mm to 1.5 mm in the subject's nail.

In an embodiment, the apparatus further includes a substance-dispenser, the substance dispenser having a nozzle, the nozzle being configured to administer the substance to a nail-bed of the subject by being placed inside the hole.

In an embodiment, the nozzle is configured to form a pressure seal with the nail by being placed inside the hole.

There is further provided, in accordance with an embodiment of the present invention, apparatus for drilling a hole in a nail of a subject, including:

a drill bit having a nail-contacting surface for contacting the subject's nail;

a force sensor for detecting a force between the nail-contacting surface and the nail; and

a drill control unit configured to:
drill a hole in the subject's nail by moving the nail-contacting surface, while the nail-contacting surface is in contact with the subject's nail, and
stop the drilling in response to the force sensor detecting that the force between the nail-contacting surface and the nail exceeds a threshold force.

There is additionally provided, in accordance with an embodiment of the present invention, apparatus for drilling a hole in a nail of a subject, including:

a drill bit having a nail-contacting surface for contacting the subject's nail;

a skin-contact sensor for detecting contact between the nail-contacting surface and a nail-bed of the subject; and

a drill control unit configured to:
drill a hole in the subject's nail by moving the nail-contacting surface, while the nail-contacting surface is in contact with the subject's nail, and
stop the drilling in response to the skin-contact sensor detecting contact between the nail-contacting surface and the subject's skin.

In an embodiment, the drill control unit is additionally configured to stop the drilling in response to the force sensor detecting that the force between the nail-contacting surface and the nail exceeds a threshold force.

In an embodiment, the skin-contact sensor includes an impedance sensor.

In an embodiment, the skin-contact sensor includes a torque sensor.

In an embodiment, the skin-contact sensor includes a force sensor.

There is further provided, in accordance with an embodiment of the present invention, a method for treating a condition of a nail of a subject, including:

placing a nail-contacting surface against a subject's nail; and

drilling a hole in the subject's nail with the nail-contacting surface, by moving the nail-contacting surface using a back-and-forth motion.

In an embodiment, drilling the hole includes relieving pressure associated with a blood-blister underneath the nail by drilling the hole in the nail.

In an embodiment, the method further includes treating onychomycosis in response to drilling the hole in the nail.

There is additionally provided, in accordance with an embodiment of the present invention, a method for treating a condition of a nail of a subject, including:

drilling a hole in the subject's nail with a nail-contacting surface;

detecting a force between the nail-contacting surface and the nail; and

stopping the drilling in response to the force exceeding a threshold.

There is further provided, in accordance with an embodiment of the present invention, a method for treating a condition of a nail of a subject, including:

drilling a hole in the subject's nail with a nail-contacting surface;

automatically detecting contact between the nail-contacting surface and a nail-bed of the subject; and

stopping the drilling in response to the detecting.

The present invention will be more fully understood from the following detailed description of embodiments thereof, taken together with the drawings, in which:

### BRIEF DESCRIPTION OF THE DRAWINGS

FIG. 1 is a schematic illustration of a nail drill, in accordance with an embodiment of the present invention;

FIG. 2 is a schematic cross-sectional illustration of the nail drill, in accordance with an embodiment of the present invention;

FIG. 3 is a schematic illustration of apparatus for performing iontophoresis, in accordance with an embodiment of the present invention;

FIG. 4 is a schematic illustration of a substance-dispenser, in accordance with an embodiment of the present invention,

FIG. 5 is a schematic illustration of an adhesive bandage, in accordance with an embodiment of the present invention; and

FIG. 6 is a schematic illustration of a finger sheath, in accordance with an embodiment of the present invention.

### DETAILED DESCRIPTION OF EMBODIMENTS

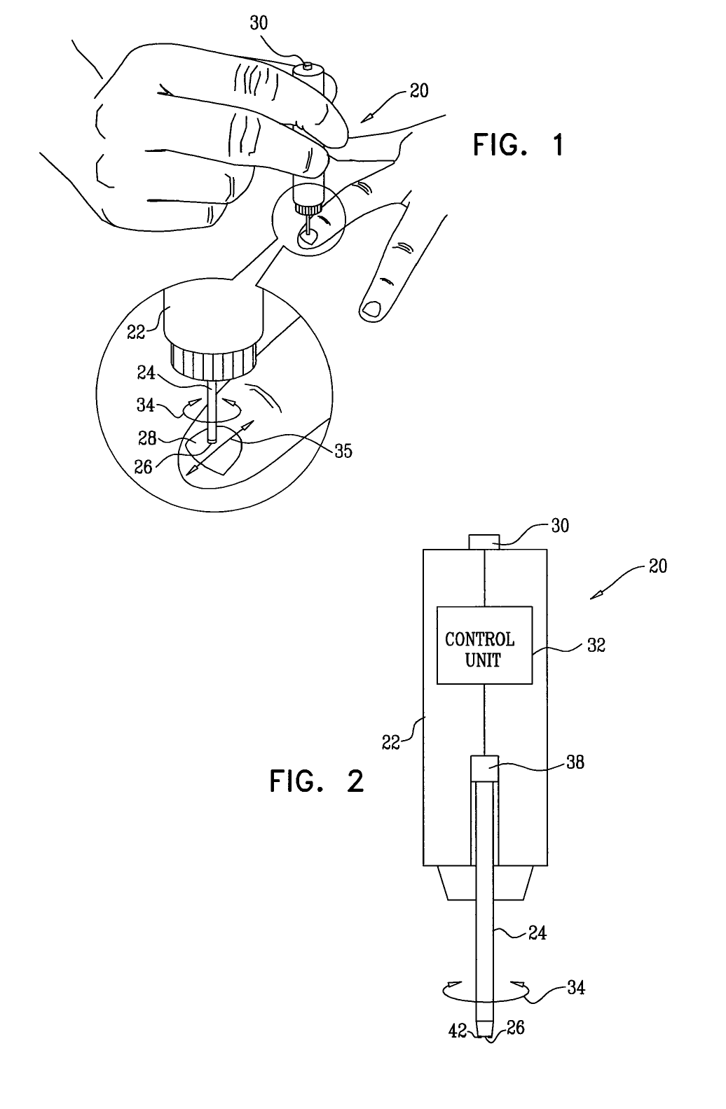

Reference is now made to FIGS. 1 and 2, which are schematic illustrations of a nail drill 20, in accordance with an embodiment of the present invention. The nail drill includes a handle portion 22 and a drill bit 24. During operation of the nail drill, a user holds the handle portion and pushes a nail-contacting surface 26 of the drill bit against a nail 28. In some embodiments, in order to activate the drill, the user presses a power button 30. Alternatively, the drill functions automatically in response to the nail exerting pressure on the nail-contacting surface. A control unit 32 drives nail-contacting surface 26 to move in a back-and-forth motion, for example, as indicated by arrow 34, or as indicated by arrow 35. Typically moving surface 26 in a back-and-forth motion ensures that upon drilling through the nail and making contact with the nail-bed, surface 26 simply moves the skin of the nail-bed, but does not drill through the skin or cause the skin any significant trauma.

In accordance with respective embodiments, nail-contacting surface 26 is pointed or flat. In some embodiments, nail-contacting surface is a flat surface that includes an abrasive material, for example, diamond powder.

For some applications, the nail contacting surface moves in the back-and-forth motion by repeatedly (a) moving in a first direction by an angle that is greater than (for example) 30 degrees, and (b) moving the nail-contacting surface in a second direction opposite to the first direction by the angle that is greater than 30 degrees. In some embodiments, the nail contacting surface moves in the back-and-forth motion by repeatedly (a) moving in a first direction by an angle that is less than 360 degrees, and (b) moving in a second direction opposite to the first direction by the angle that is less than 360 degrees. Typically, the nail-contacting surface executes 2-60 cycles of the back-and forth motion per second. For some applications, the nail-contacting surface executes a number of cycles of the back-and forth motion per second that is greater than 60. In some embodiments, the control unit controls the back-and-forth motion electronically. Alternatively, gearing is used to facilitate the back-and-forth motion.

For some applications, one to twelve holes are drilled in the nail. Typically, the holes are drilled near to the base of the nail.

In some embodiments, as an alternative or in addition to moving the nail-contacting surface in the back-and-forth motion, the nail drill includes one or more sensors, for example, a torque or force sensor 38, or an impedance sensor (which typically comprises two or more electrodes on nail-contacting surface 26). In some embodiments, the sensor acts to detect when nail-contacting surface 26 makes contact with the nail-bed underlying the nail. For example, the torque of drill bit 24 decreases when it breaks through the nail and comes into contact with elastic tissue of the nail-bed. In response to the sensor detecting the decrease in torque, control unit 32 stops the drilling. For some applications, in response to force sensor 38 detecting that the nail drill is being pushed by the user toward the nail with a force that exceeds a threshold force, the control unit stops the drilling.

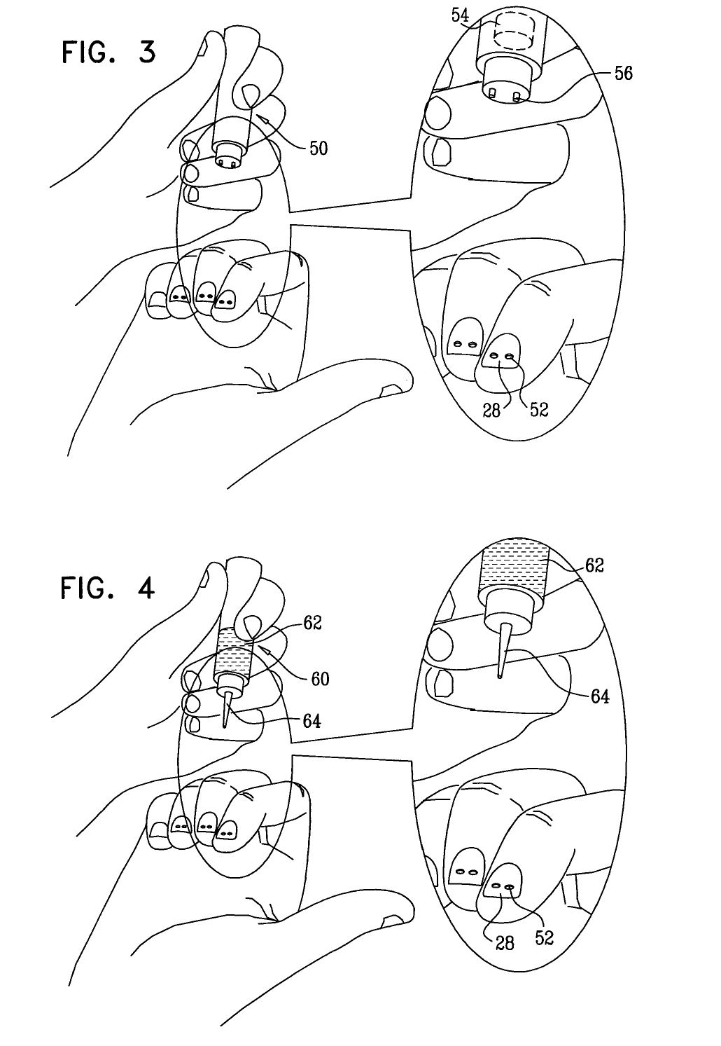

Reference is now made to FIG. 3, which is a schematic illustration of iontophoresis apparatus 50 for administering a substance to the subject by performing iontophoresis, in accordance with an embodiment of the present invention. In some embodiments, nail drill 20 drills two or more holes 52 in a subject's nail 28. A substance (for example, substance 62 described hereinbelow, or another substance) is administered to the nail-bed underlying the nail. In some embodiments, an iontophoresis control unit 54 drives a current via two or more electrodes 56, in order to iontophoretically drive the substance into tissue of the nail-bed. Typically, the subject drills the holes in a pattern that matches the pattern of the array of electrodes on iontophoresis apparatus 50. For example, the subject may be provided with an adhesive guide for placing on the nail before the subject begins to drill the nail. The adhesive guide contains marking indicating the locations where the subject should drill. The markings are arranged such that the pattern of drilled holes will match the array of electrodes 56 on iontophoresis apparatus 50. It is noted that although 2×1 arrays of electrodes 56 and holes 52 are shown in FIG. 3, the scope of the present invention includes using different arrays of electrodes and holes. For example, an array of one to twelve holes may be drilled.

In some embodiments, nail drill 20 is configured to drill circular holes 52 in nail 28, the holes each having a diameter of 0.1 mm to 2.0 mm, e.g., 0.5 mm to 1.5 mm.

Reference is now made to FIG. 4, which is a schematic illustration of a substance dispenser 60 for administering a substance to the subject, in accordance with an embodiment of the present invention. Typically the dispenser dispenses a substance 62 for treating onychomycosis, for example, itraconazole (marketed as Sporanox®), and/or terbinafine (marketed as Lamisil®). In some embodiments, substance 62 includes antifungal agents and antibacterial agents for treating onychomycosis. Substance dispenser 60 includes a nozzle 64 for placing inside holes 52 on the subject's nail and administering the substance to the underlying nail-bed. In some embodiments, the holes are drilled in a specific size, and the nozzle is sized to match the size of the holes.

For some applications, holes 52 and nozzle 64 are sized such that when the nozzle is placed inside one of the holes, the nozzle forms a pressure seal with the nail. Subsequently, when the substance is dispensed from the dispenser, the substance is forced underneath the subject's nail. For some applications, the diameter of each hole 52 is 1-2 mm.

In some embodiments, substance 62 is administered to the subject via the holes using a different technique. For example, drill bit 24 may administer substance 62, through a lumen in the drill bit, via the holes, or a syringe may be used to inject the substance under pressure into a hole. Alternatively or additionally, the substance is sprayed onto the nail, such that the substance enters the holes.

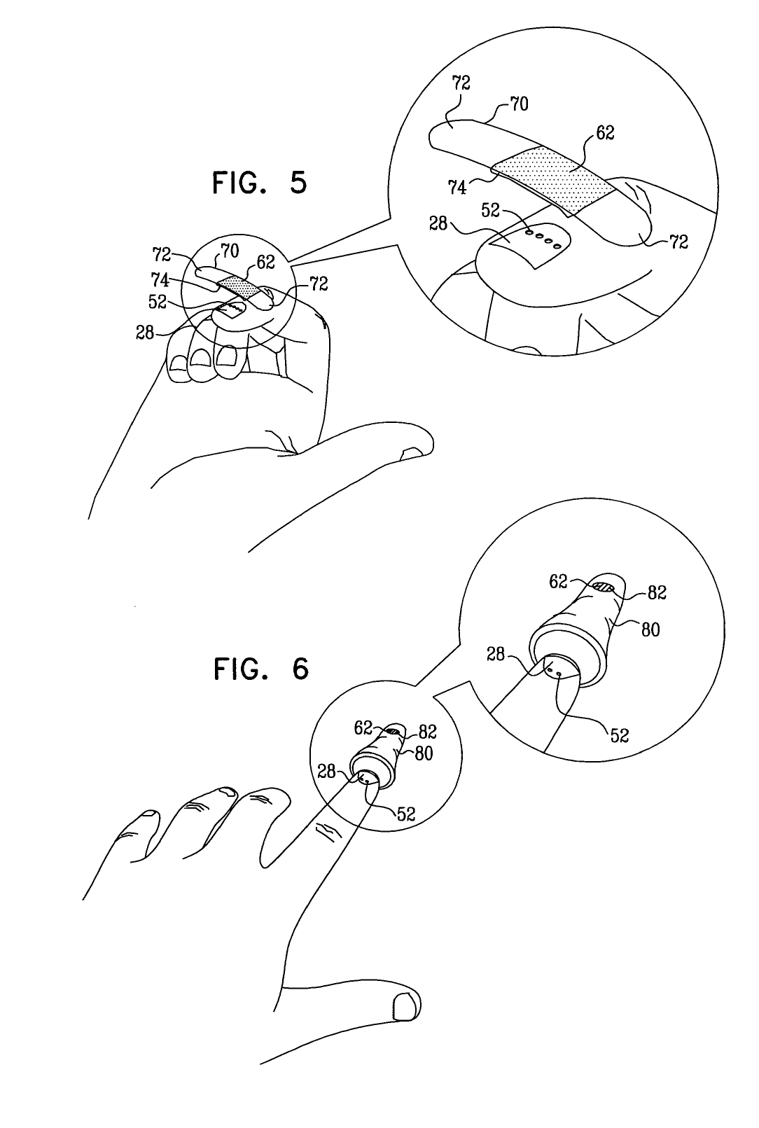

Reference is now made to FIG. 5, which is a schematic illustration of an adhesive bandage 70, in accordance with an embodiment of the present invention. The bandage contains adhesive portions 72, and a central substance-containing portion 74. In some embodiments, after the subject has drilled holes 52 in nail 28, the subject administers substance 62 to the underlying nail-bed by adhering portions 72 to the finger such that substance-containing portion 74 is positioned over the holes.

Reference is now made to FIG. 6, which is a schematic illustration of a finger sheath 80, in accordance with an embodiment of the present invention. The finger sheath contains a substance-containing portion 82 inside the sheath, and portion 82 contains substance 62. In some embodiments, subsequent to drilling holes 52 in the subject's nail, the subject places the sheath over his/her finger in order to administer the substance to the nail-bed that underlies the holes. The sheath is placed such that substance-containing portion 82 is positioned over the holes.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for drilling a hole in a nail of a subject, comprising:
a drill bit having a nail-contacting surface for contacting the subject's nail; and
a drill control unit configured to drill a hole in the subject's nail by moving the nail-contacting surface in a back-and-forth motion, while the nail-contacting surface is in contact with the subject's nail.

2. The apparatus according to claim 1, wherein the nail-contacting surface is flat.

3. The apparatus according to claim 1, wherein the control unit is configured to execute 2-60 cycles of the back-and forth motion per second.

4. The apparatus according to claim 1, further comprising a skin-contact sensor for detecting contact between the nail-contacting surface and a nail-bed of the subject.

5. The apparatus according to claim 1, further comprising a substance for treating onychomycosis, two or more electrodes, and an iontophoresis control unit,
wherein the drill control unit is configured to drill two or more holes in the subject's nail, and
wherein the iontophoresis control unit is configured to iontophoretically drive the substance into a nail-bed of the subject by driving the electrodes to drive a current via the holes.

6. The apparatus according to claim 1, wherein the drill control unit is configured to move the nail-contacting surface in the back-and-forth motion by repeatedly (a) moving the nail-contacting surface in a first direction by an angle that is greater than 30 degrees, and (b) moving the nail-contacting surface in a second direction opposite to the first direction by the angle that is greater than 30 degrees.

7. The apparatus according to claim 1, wherein the drill control unit is configured to move the nail-contacting surface in the back-and-forth motion by repeatedly (a) moving the nail-contacting surface in a first direction by an angle that is less than 360 degrees, and (b) moving the nail-contacting surface in a second direction opposite to the first direction by the angle that is less than 360 degrees.

8. The apparatus according to claim 1, further comprising anti-fungal and anti-bacterial agents for administering to a nail-bed of the subject via the hole.

9. The apparatus according to claim 1, further comprising a finger sheath that contains a substance for treating onychomycosis, the sheath being configured to administer the substance to a nail-bed of the subject via the hole, by being placed on the subject's finger over the hole.

10. The apparatus according to claim 1, further comprising an adhesive bandage that contains a substance for treating onychomycosis, the bandage configured to administer the substance to a nail-bed of the subject via the hole, by being adhered to the subject's finger over the hole.

11. The apparatus according to claim 1, wherein the apparatus is configured to drill a hole having a diameter of 0.1 mm to 2.0 mm in the subject's nail.

12. The apparatus according to claim 11, wherein the apparatus is configured to drill a hole having a diameter of 0.5 mm to 1.5 mm in the subject's nail.

13. The apparatus according to claim 1, further comprising a substance-dispenser, the substance dispenser having a nozzle, the nozzle being configured to administer the substance to a nail-bed of the subject by being placed inside the hole.

14. The apparatus according to claim 13, wherein the nozzle is configured to form a pressure seal with the nail by being placed inside the hole.

15. (canceled)

16. Apparatus for drilling a hole in a nail of a subject, comprising:
a drill bit having a nail-contacting surface for contacting the subject's nail;
a skin-contact sensor for detecting contact between the nail-contacting surface and a nail-bed of the subject; and
a drill control unit configured to:
drill a hole in the subject's nail by moving the nail-contacting surface, while the nail-contacting surface is in contact with the subject's nail, and
stop the drilling in response to the skin-contact sensor detecting contact between the nail-contacting surface and the subject's skin.

17. The apparatus according to claim 16, wherein the drill control unit is additionally configured to stop the drilling in response to the force sensor detecting that the force between the nail-contacting surface and the nail exceeds a threshold force.

18-20. (canceled)

21. A method for treating a condition of a nail of a subject, comprising:
placing a nail-contacting surface against a subject's nail; and
drilling a hole in the subject's nail with the nail-contacting surface, by moving the nail-contacting surface using a back-and-forth motion.

22. (canceled)

23. The method according to claim 21, further comprising automatically detecting contact between the nail-contacting surface and a nail-bed of the subject.

24-25. (canceled)

26. The method according to claim 21, wherein drilling the hole comprises moving the nail-contacting surface using the back-and-forth motion by repeatedly (a) moving the surface in a first direction by an angle that is greater than 30 degrees, and (b) moving the surface in a second direction opposite to the first direction by the angle that is greater than 30 degrees.

27. The method according to claim 21, wherein drilling the hole comprises moving the nail-contacting surface in the back-and-forth motion by repeatedly (a) moving the surface in a first direction by an angle that is less than 360 degrees, and (b) moving the surface in a second direction opposite to the first direction by the angle that is less than 360 degrees.

28-36. (canceled)

37. A method for treating a condition of a nail of a subject, comprising:
drilling a hole in the subject's nail with a nail-contacting surface;
automatically detecting contact between the nail-contacting surface and a nail-bed of the subject; and
stopping the drilling in response to the detecting.

38-40. (canceled)
